(19) United States
(12) Patent Application Publication
(10) Pub. No.: US 2022/0331473 A1
(43) Pub. Date: Oct. 20, 2022

(54) UV SANITATION SYSTEMS AND METHODS

(71) Applicant: UVC Cleaning Systems Inc., McMurray, PA (US)

(72) Inventors: Robert L. Gilling, Caro, MI (US); Robert C. Gilling, Caro, MI (US); Steve F. Willenborg, Campbell, CA (US); David S. Schlafman, McMurray, PA (US)

(21) Appl. No.: 17/723,597

(22) Filed: Apr. 19, 2022

Related U.S. Application Data
(60) Provisional application No. 63/176,800, filed on Apr. 19, 2021.

(51) Int. Cl. A61L 9/20; G07C 9/00

(52) U.S. Cl. CPC A61L 9/20; G07C 9/00563; A61L 2209/111; A61L 2209/12

(57) ABSTRACT

A method for sanitizing a space includes positioning a plurality of light sources configured to emit light in a wavelength between 185 nm and 405 nm. At least one pathogen which is desired to kill in the space is determined. A contamination level of the pathogen in the space is determined with at least one sensor. A treatment plan is determined for emitting light from at least one of the plurality of light sources with a controller to eliminate the pathogen. The treatment plan includes a determination of at least one light wavelength in the range of 185 nm and 405 nm, at least one light intensity, and at least one duration of time at which to emit the light to kill the pathogen. Light is emitted in accordance with the treatment plan.

### CROSS-REFERENCE TO RELATED APPLICATIONS

This application claims the benefit of U.S. Provisional Patent Application No. 63/176,800, file Apr. 19, 2021, entitled “UV SANITATION SYSTEMS AND METHODS,” The entire disclosure of which is incorporated by reference in its entirety.

### TECHNICAL FIELD

The present invention relates to a sanitation system incorporating at least one source of UV light for residential and/or commercial environments.

### BACKGROUND

This section provides background information related to the present disclosure which is not necessarily prior art.

The general public has recently become more aware than ever about the benefits of disinfectants. One of the major driving forces of the recent insurgence of awareness has been the worldwide pandemic caused by COVID-19. COVID-19 is a strain of the coronavirus that effects the respiratory tract leading to a range of symptoms from minor to lethal. This increased awareness paralleled the demand for disinfectants for both residential and commercial uses. There are a number of disinfecting technologies ranging from topical liquid sanitizers to light sources that generate a spectrum of electromagnetic radiation at various wavelengths and frequencies.

One popular type of electromagnetic radiation is ultraviolet (UV light). UV light contains several unique properties and characteristics, which can be used to disinfect up to 99.9% of viruses, bacteria, mold, spores and other harmful microbes. UV light falls within the general wavelength range of 10 nm to 405 nm. One type of UV light, UV-C, is capable of killing tuberculosis, E. coli, methicillin-resistant S. aureus (MRSA), coronavirus strains and more. Light sources operating within the UV-C spectrum, i.e. UV-C rays within the wavelength range of 200 nm to 280 nm (optimized at 235.7 nm, i.e., approximately 254 nm), have been shown to deactivate the cellular ribonucleic acid (RNA) and deoxyribonucleic acid (DNA), as well as the reproductive capabilities of microorganisms. Although highly effective in eliminating microbes on surfaces and in the air, the 200 nm to 280 nm UV-C range can be dangerous to humans. More particularly, extended exposure to this UV-C range, typically using fluorescent UV lamps, can cause mild to severe skin damage and temporary to permanent vision loss without proper safety gear.

Advancements in UV light technology has addressed some of the drawbacks and limitations of traditional UV-C sanitation by introducing far-UV sanitation light sources into the market. This type of light source generally utilizes 222 nm UV range (or spectrum) rays to eliminate bacteria, viruses, mold, spores, fungi and other deadly microbes, but is considered to be eye and skin safe for humans as 222 nm UV cannot penetrate the skin to levels that cause burns or severe damage. More particularly, studies have shown that prolonged exposure to irradiation with 222 nm range far-UV light sources does not cause epidermal lesions that are associated with exposure to traditional 254 nm UV-C rays. This finding, among others, has revolutionized the application of UV-C disinfection, allowing light sources to be deployed safely in busy locations, occupied areas, manufacturing floors, restaurants, public buildings, mass transit areas and more. Far-UV light sources typically include a mercury-free 222 nm energy source with quartz glass are the artificial light source of choice when implementing far-UV disinfection on surfaces.

There are a myriad of factors that must be taken into consideration when administering UV-C light—both optimized 254 nm and far-UV 222 nm—to eliminate viruses or bacteria. These factors include: light intensity, duration of treatment, distance between the UV light source and object or surface, obstructions and environment (temperature and humidity). To ensure effective UV disinfection, different types of far-UV devices have been utilized. For example, a portable sanitation cart that emits a full 360 degree 222 nm UV beam is ideal for sanitizing large rooms or facilities, while a handheld UV-C lamp may be implemented for cleaning objects or hard-to-reach surfaces and equipment. A compact far-UV sanitation box may also be used to disinfect personal devices and tools on the field or at facilities. Far-UV devices are ozone-free and are also capable of disinfecting air in occupied spaces.

As such, there is a continuing desire to develop far-UV devices to reduce the spread of germs.

### SUMMARY OF THE DISCLOSURE

This section provides a general summary of the disclosure and should not be interpreted as a complete and comprehensive listing of all the objects, aspects, features and advantages associated with the present disclosure.

According to an aspect of the disclosure, a method is provided for sanitizing a space. The method includes positioning a plurality of light sources in the space. Each of the light sources are configured to emit ultraviolet light in a wavelength range between 185 nm and 405 nm. The method also includes determining at least one pathogen which is desired to kill in the space. The method also includes determining a contamination level of the pathogen in the space with at least one sensor. The method also includes determining a treatment plan for emitting light from at least one of the plurality of light sources with a controller to eliminate the at least one determined pathogen based on the determined contamination level, wherein the treatment plan includes a determination of at least one wavelength in the range of 185 nm and 405 nm at which to emit the light, at least one intensity at which to emit the light, and at least one duration of time at which to emit the light from the at least one of the plurality of light sources to kill the pathogen. The method also includes emitting light in the space from the at least one of the plurality of light sources in accordance with the treatment plan.

Accordingly, the disclosure provides a method for efficiently killing a predetermined pathogen, and the method is easily adaptable based on changing factors to provide improvements in disinfecting efficiency and safety.

According to another aspect of the disclosure, another method for sanitizing a space includes positioning at least one light source in a space. The at least one light source is configured to emit ultraviolet light in a wavelength range between 185 nm and 405 nm. The method also includes emitting light in the space from the at least one light source at a first wavelength in the range between 185 nm and 405 nm. The method also includes detecting a condition in the space with a sensor. The method also includes emitting light in the space from the at least one of the plurality of lights sources at a second wavelength that is different than the first wavelength in the range between 185 nm and 405 nm in response to the detection of the condition in the space with the sensor.

Accordingly, the method provides a means of quickly changing a wavelength at which UVC light is emitted to adapt to changing conditions, such as safety or an amount of a contaminant in the space. For example, the system may be used to track a person or object moving to different locations in the space, and to adjust the light sources accordingly to provide improved safety. As another example, the system may be configured to adjust its output based on a new detected level of particulate in the space.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1 is a schematic view of a sanitation system including a plurality of light sources;

FIG. 2 is a schematic view of a light source that includes a circulation unit and a distribution element;

FIG. 3 is a schematic view of an operating circuit of the sanitation system;

FIG. 4 is a flow diagram of a first example method of using the sanitation system;

FIG. 5 is a flow diagram of a second example method of using the sanitation system;

FIG. 6 is a flow diagram of a third example method of using the sanitation system;

FIG. 7 is a flow diagram of a fourth example method of using the sanitation system; and

FIG. 8 is a flow diagram of a fifth example method of using the sanitation system.

### DETAILED DESCRIPTION

Example aspects will now be described more fully with reference to the accompanying drawings. In general, the subject aspects are directed to a sanitation system incorporating at least one source of UV light for residential and/or commercial environments (indoors and outdoors). However, the example embodiments are only provided so that this disclosure will be thorough, and will fully convey the scope to those who are skilled in the art. Numerous specific details are set forth such as aspects of specific components, devices, and methods, to provide a thorough understanding of aspects of the present disclosure. It will be apparent to those skilled in the art that specific details need not be employed, that example aspects may be embodied in many different forms and that neither should be construed to limit the scope of the disclosure. In some example aspects, well-known processes, well-known device structures, and well-known technologies are not described in detail.

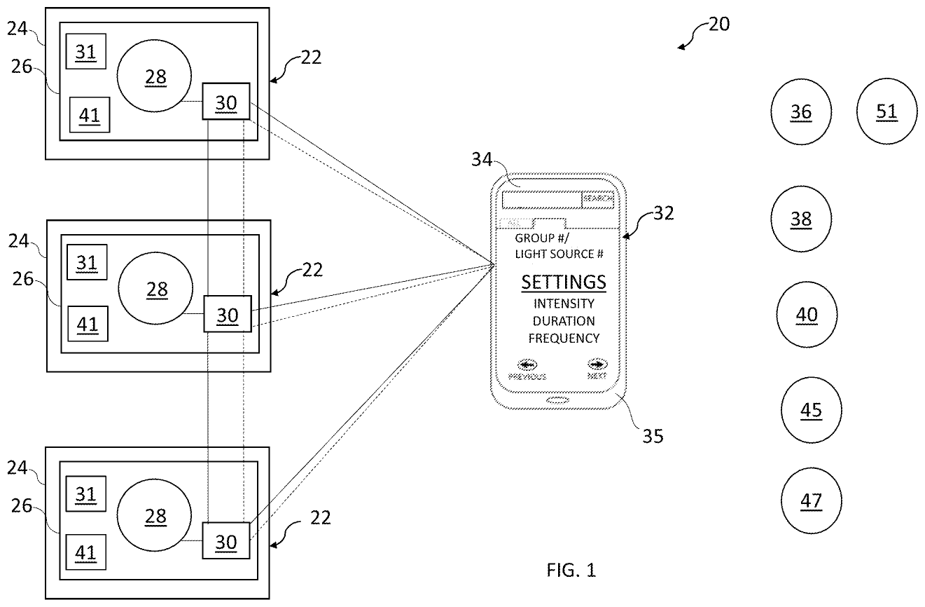

Referring to the figures, wherein like numerals indicate corresponding parts throughout the several views, a sanitation system 20 is generally shown in FIG. 1. The sanitation system 20 includes at least one light source 22, which may utilize UV light in the wavelength range of 185 nm and 405 nm. Specific ranges may be selected for specific purposes. For example, 185 nm light may be selected for ozone disinfection purposes, and 222 nm and 253.7 nm (i.e., approximately 254 nm) may be selected for killing specific predetermined pathogens. For example, the at least one light source may include a UV light having a mercury-free excimer with quartz glass and may operate in optimized approx. 254 nm and far-UV 222 nm. The at least one light source 22 may include a housing 24, a lens cover 26 connected to the housing 24, and at least one lamp 28 located within a space defined by the housing 24 and the lens cover 26. A control circuit 30 may be in communication the lamp 28 for controlling various operations thereof. The control circuit 30 may be remote or local to the at least one light source 22. In some embodiments, the control circuit 30 is local and can be adjusted manually at each light source 22 (e.g., on/off, intensity, frequency, time duration, sensor activation, etc.). In some embodiments, the control circuit 30 is local and in a wired or wireless communication with a moderator circuit 32 that sends instructions to the control circuit 30. In some embodiments, the at least one light source includes a plurality of light sources 22 each, or in predetermined groupings, having a control circuit 30 that are in communication with the moderator circuit 32 for performing certain functionalities with select light sources 22 or groupings of light sources 22. In some embodiments, the at least one light source includes a plurality of light sources 22 each, or in predetermined groupings, having a control circuit 30 that are in communication with a control circuit 30 of another individual or predetermined grouping of light sources 22 for providing certain functionalities in concert. Communication between control circuits 30 may be via a mesh network or other suitable network. In some embodiments, the moderator circuit 32 is associated with a computer, tablet, or mobile device. The various light sources 22 may be located in a single room, throughout a building, facility, campus, in one or more vehicles, or across multiple locations. The predetermined groupings of light sources 22 may be dependent on proximity or environmental characteristics such as occupation status or germ risk level associated with types of work or conditions. The light sources 22 each may include a supplementary source of light 31 for emitting a predetermined amount of visible light.

With continued reference to FIG. 1, the moderator circuit 32 may include a user interface 34 for selecting predetermined groupings of light sources 22 or individual light sources 22 and controlling (manual or automated) certain operational conditions such as intensity, duration of illumination, wavelength, frequency, motion sensing protocols (e.g., presence and proximity), light sensing protocols, environmental sensing protocols, dosing protocols, or a combination thereof. The user interface 34 may be presented on various devices including, but not limited to, a mobile phone 35, tablet, personal computer, handheld unit, etc. In accordance with the above, the sanitation system 20 may include various sensors including, but not limited to, motion sensors 36, light sensors 38, particulate sensors 45, radiometric energy sensors 47, environmental sensors 40 (e.g., humidity or temperature sensors), a space size sensor 51, other sensors, or a combination thereof.

In some embodiments, each or select light sources 22 may have various shapes, configurations, and assemblies. For example, in some embodiments, each or select light sources 22 may be configured as prepackaged 2′×2′ troffer-style 222 nm and/or 253.7 nm emitters or prepackaged 2′×4′ troffer-style 222 nm and/or 253.7 nm emitters. In some embodiments, each or select light sources 22 may be configured to be moveable between locations or as a fixture on a ceiling, wall, or floor in a residential or commercial environment. For example, each or select light sources 22 may be retrofitted or otherwise located in recesses of the ceiling (e.g., a solid ceiling or a drop ceiling), wall, or floor in new or old constructions. In some embodiments, the light sources 22 located in recesses may be removable, for example, the housing 24 may be removable or the lamp 28 may be removable for service or selective replacement with a non-sanitizing light source. Removable portions of the light source 22 can be selectively held in place by springs, clips, collapsible wires, screws, tape, or combinations thereof. In some embodiments, each or select light sources 22 may be free standing on a support (not shown). In some embodiments, one or more of the light sources 22 are positioned on a mobile cart to facilitate easy movement of the light sources 22 to different locations. In other embodiments, one or more of the light sources 22 are fixed. In some embodiments, each or select light sources 22 may be configured as a can-type light.

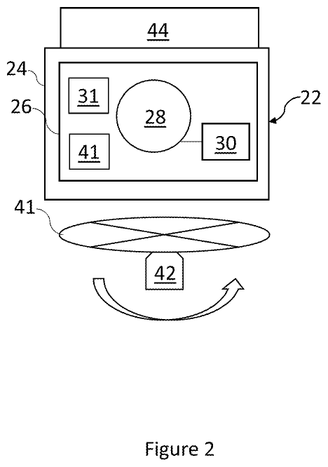

With reference now to FIG. 2, the sanitation system 20 may include a light distribution element 41. The light distribution element 41 may include an internal surface of the housing 24 that is reflective to improve array distribution and 222 nm and 253.7 nm (approx. 254 nm) efficacy. The light distribution element 41 may further include portions of the lens cover 26, which may include directional and expanded 222 nm and 253.7 nm (approx. 254 nm) energy diffusion patterns to array distribution and increase efficacy. The light distribution element 41 may further be an independent element within or moveable within the illumination array of the lamp 28. In some embodiments, the lens cover 26 with the diffusion pattern may further be selectively interchangeable. In some embodiments, one or more of the reflective internal surface of the housing 24, the lens cover 26, or other associated elements with the light distribution element 41 may define three-dimensional patterns such as perforations, pyramids, cones, hexagons. The three-dimensional patterns may be located within the array of illumination of the light source 22 at a fixed, veering, or otherwise moving distance or position to reflect and/or refract the UVC light or dispersion pattern onto select portions or patterns of the residential or commercial environment. Movement of the three-dimensional patterns may be by operation of the control circuit 30, the moderator circuit 32, at least one of the sensors, or a combination thereof via mechanical devices such a gear units 42. In some embodiments, the three-dimensional patterns may further define louvered or open shape perforations, fins, open shapes, curved objects, straight, pyramid, cone, hexagon, other shapes, or combinations thereof for even dispersion and/or changing the array pattern, size of the array pattern, direction of the array pattern, or a combination thereof. In some embodiments, the three-dimensional patterns may be modified based on light detection with the light sensors 38, for example, to ensure appropriate coverage and intensity along a sloped ceiling or other surface.

The sanitation system 20 may further include a circulation unit 44 or a plurality of circulation units 44 that controls air flow within the residential or commercial environment. For example, in some embodiments, the circulation unit 44 may located in or on the housing 24 or remote from the housing 24 and guide air within the array of light. In some embodiments, the circulation unit 44 is configured to guide air within and through the housing 24. In some embodiments, the circulation unit 44 includes a fan. In some embodiments, the circulation unit 44 can be moved to change a direction of the air flow. The speed, movement, and operation may be controlled via operation of the control circuit 30, moderator circuit 32, at least one of the sensors, or a combination thereof.

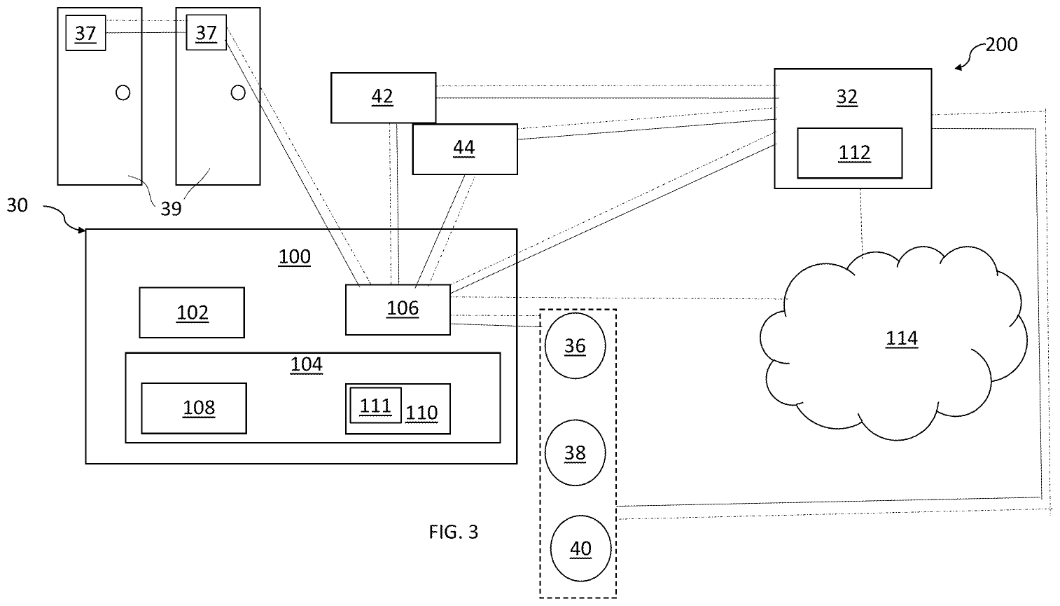

With reference now to FIG. 3, a schematic view of the sanitation system 20 is illustrated. As described, the sanitation system 20 includes a control circuit 30, a moderator circuit 32. The various elements provided therein allow for a specific implementation. Thus, one of ordinary skill in the art of electronics and circuits may substitute various components to achieve a similar functionality. The control circuit 30 may include a controller 100 having a processor 102 and a memory 104, such as in table 111. The processor 102 may include any suitable processor, such as those described herein. Additionally, or alternatively, the controller 100 may include any suitable number of processors, in addition to or other than the processor 102. The memory 104 may comprise a single disk or a plurality of disks (e.g., hard drives), and includes a storage management module that manages one or more partitions within the memory 104. In some embodiments, memory 104 may include flash memory, semiconductor (solid state) memory or the like. The memory 104 may include Random Access Memory (RAM), a Read-Only Memory (ROM), or a combination thereof. The memory 104 may include instructions that, when executed by the processor 102, cause the processor 102 to, at least, perform the systems and methods described herein. The controller 100 may further include a communications module 106 in wired and/or wireless communication with the series of sensors, the moderator circuit 32, and other control circuits 38. Programs and/or software 108 and light profile settings 110 (e.g., intensity, duration, and wavelength for killing certain types of contaminants associated with certain areas) are saved on the memory 104, such as via a pathogen table. The moderator circuit 32 may also include a controller 112, which may include each of the same features, elements, and functionalities of the controller 100. The sanitation system 20 may also include a network 114 in communication with the control circuit 38 and/or moderator circuit 32 such that software updates can be updated via a transmission of information from the network 114.

In some embodiments, the sanitation system 20, the control circuit 30, and/or the moderator circuit 32 may perform the methods described herein. However, the methods described herein are not meant to be limiting, and any type of software executed on a controller or processor can perform the methods described herein without departing from the scope of this disclosure. For example, a controller, such as a processor executing software within a computing device, can perform the methods described herein.

As will be discussed in more detail below, in some embodiments, the systems and methods described herein may be configured to sense a presence of an occupant (e.g., human or animal) with the motion sensor 36 and, in response, generate a signal to modify a light source 22, a plurality of light sources 22, or a select grouping of light sources 22. For example, modify the intensity, duration, direction, frequency, array pattern, wavelength, on/off status, or combinations thereof. For example, the system may be configured to deactivate at least one of the light sources 22 in response to a detection of an occupant in the space with the motion sensor 36 to provide improved safety for the occupant, especially from light that falls within a wavelength that is harmful to human skin. Alternatively, the system may be configured to switch a wavelength of light emitted from one or more of the light sources 22 from a first wavelength to a second wavelength (e.g., from 253.7 nm (approx. 254 nm) to 222 nm) in response to detection of an occupant with the motion sensor 36 in order to provide improved safety for the occupant. As part of this operation, the system may be configured to only deactivate a light source 22 in a region of the space at which the occupant was detected, while leaving one or more light sources 22 on in other regions of the space where the occupant is not located. It should be appreciated that a series of motion sensors 36 may be provided in different regions of the space to track where occupants are located and actively activate/deactivate or change wavelengths and/or other parameters based on current occupant locations. Indeed, the controller 100 may be configured to actively activate/deactivate and change wavelengths and/or other parameters of the light source 22 based on current needs any number of teams during a treatment session in order to optimize the treatment.

In some embodiments, the system may further include one or more electronic door locks 37 associated with one or more doors 39 that permit ingress and egress of occupants into the space in which the one or more light sources 22 are located. More particularly, the system may be configured to activate the electronic door locks 37 in response to a detection of light (such as with the light sensor 38) being emitted from the at least one light source 22 at a predetermined wavelength range in order to prevent occupants from entering the room to provide improved safety. For example, the electronic door locks 37 may be activated in response to a detection of light at a wavelength of 253.7 nm (approx. 254 nm). The electronic door locks 37 may be deactivated in response to the sensor 38 detecting that light falls outside of the predetermined wavelength range, or generally in a safe range. Any number of sensors 38 could be positioned in the space and any number of electronic door locks 37 may be provided to allow only at risk places in the space to be locked.

In some embodiments, the systems and methods described herein may be configured to sense an environmental condition, such as a temperature or humidity (such as with environment sensors 40) and, in response, generate a signal to modify a light source 22, a plurality of light sources 22, or a select grouping of light sources 22. For example, modify the intensity, frequency, duration, direction, array pattern, wavelength, on/off status, or combinations thereof.

In some embodiments, the systems and methods described herein may be configured to sense a light, such as with light sensor 38, (e.g., a UV light intensity in a residential or commercial environment) and, in response, generate a signal to modify a light source 22, a plurality of light sources, or a select grouping of light sources. For example, modify the intensity, of the light source 22, run a diagnostic check of the light source 22, request service of a light source 22, or combinations thereof.

In some embodiments, the systems and methods described herein may be configured to upon a scheduled condition (e.g., before or after a work schedule, shift schedule, time of day, day of week, the passage of a scheduled amount of time, etc.), generate a signal to modify a light source 22, a plurality of light sources 22, or a select grouping of light sources 22. For example, modify the intensity, frequency, duration, direction, array pattern, wavelength, on/off status, or combinations thereof. As part of this, the light sources 22 may have an internal clock with memory for managing an illumination schedule. The light sources 22 may be configured to operate at different wavelengths, intensities and for different durations of time multiple times in a single day. For example, the light sources 22 may be configured to activate at 253.7 nm (approx. 254 nm) while under a schedule that correlates with when occupants are not present in the location of the light sources 22. The light sources may also be configured to activate at 222 nm when occupants are present.

In some embodiments, the light sources 22 may include a temperature sensor 41 for determining a temperature of the light source 22. The controller 100 may be configured to deactivate the light source 22 in response to a detection that the light source 22 has exceeded a predetermined temperature.

In some embodiments, the wireless connections may electrically connect the light sources 22 to quicken disinfection cycle time. The light sources 22 may or may not all stay on until the last light source 22 is turned off/on. The light sources 22 may or may not have multiple spectrums of UV-C light placed throughout the environment. The series of sensors may or may not override the preset timing functions to achieve a quicker cycle time. In some embodiments, the light sources 22 may or may not be connected to show diagnostic information including a time stamp. This includes on/off time, individual light source 22 or grouping run time, motion indicator settings, ballast power settings, meshing or grouping units together, setting cycle schedules of intensity, etc. In some embodiments, the light sources 22 may or may not have controls permanently mounted to one or more fixtures including the housing 24 or nearby walls, ceilings, or floors. In some embodiments, the systems and methods herein may control timing, motion or power, of more than one UV spectrum. In some embodiments, the systems and methods herein may control on/off or delay due to motion in the 222 UV-C spectrum. In some embodiments, the systems and methods herein may control a timing function of the on/off, which may be determined by integrated motion sensors that are located on/and or near the door or other locations in the environment. In some embodiments, the light source 22 may include blended light spectrums with 222 nm and another light spectrum such as 264 nm and/or 254 nm as controlled by the systems and methods.

In some embodiments, the systems and methods herein may control the 222 nm light source 22 by way of timer/App/Wi-Fi/BT system, Networking and Internet-based product configuration. In some embodiments, the light source 22 may include one or both 222 nm and approx. 254 nm light sources 22. The timing function of the on off or dimming can be determined by integrated motion sensors 36 that are located on/and or near the door, room, and/or building.

In some embodiments, the systems and methods may include both a light source 22 and a supplemental light 31 purely for illumination (e.g., florescent, LED), which may be in the same housing 24 and controlled by the systems and methods. In some embodiments, the light sources 22 may be fixed into a structure like a building or on one or more mobile units like a cart, skid, or similar structure. In some embodiments, the light source 22 may include a replaceable structure, such as a lamp 28. In some embodiments, the systems and methods may include a power connection including at least one of a lamp socket (e.g., the base of a light bulb to connect to a power source or UL approved electrical connector), back-up generators, batteries, other power sources, or combinations thereof. In some embodiments, the systems and methods may include an inverter inside or outside the light source 22 that varies the voltage from 120/240 input down to 24V in view of the conditions described above. In some embodiments, the light sources 22 may be pre-packaged units in a 2′×2′ or 2′×4′ ceiling tile or any unit made to fit standard commercial and residential approved lighting fixture. In some embodiments, the light sources 22 may include more than one lamp including a combination of illumination lighting and spectrums of UV-C lighting.

As previously noted, in some embodiments, settings of the light sources 22 may be accessed and managed via a mobile device 43. The mobile device 43 may be programmed with various operator profiles for tracking and saving the operator's selections. Furthermore, certain profiles may be programmed to have limited access to certain features.

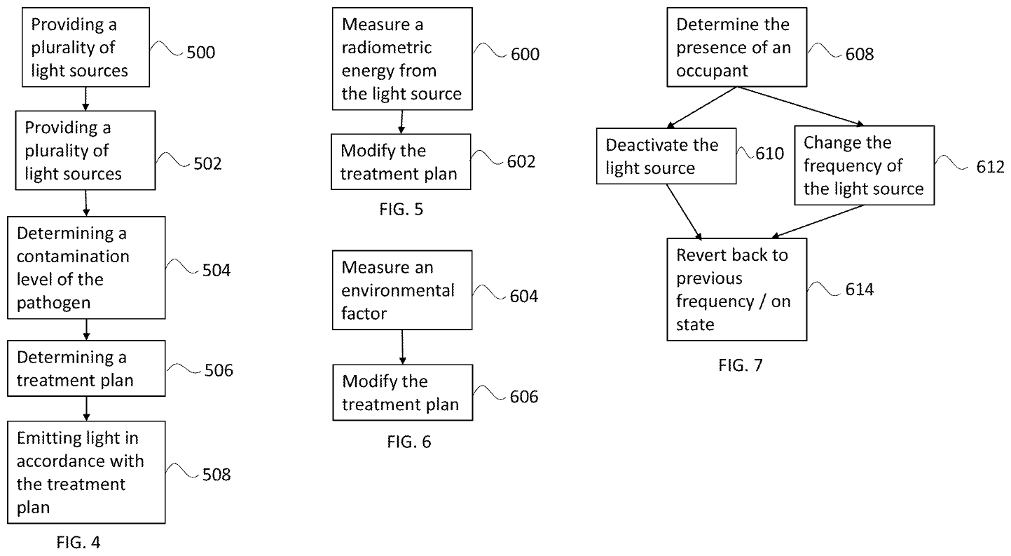

In accordance with the above, and with reference to FIG. 4, a method for sanitizing a space, such as a room in a building or an interior of a vehicle is provided. The method includes 500 providing a plurality of light sources 22 in the space, wherein the plurality of light sources 22 are configured to emit ultraviolet light in a wavelength range between 185 nm and 405 nm. The method also includes 502 determining at least one pathogen which is desired to kill in the space. For example, the pathogen may include various viruses and bacterias. It should be appreciated that the controller 100 may be configured to pull pathogen specific data from the light profile settings 110 (e.g., intensity, duration, wavelength, frequency for killing certain types of contaminants associated with certain areas) such as in the associated pathogen table 111 contained in the controller's 100 memory 104. For example, the pathogen table 111 may include information associated with a required amount of energy required to kill the pathogen based on amounts of the pathogen present. The method also includes 504 determining a contamination level of the pathogen in the space with the at least one sensor (such as particulate sensor 45). For example, the sensor 45 may be configured to measure a level of particulate associated with the pathogen in air in the space. For example, the sensor 45 may detect that the particulates of the pathogen fall in a range from 0.1 to 2.5 microns. The method continues with 506 determining a treatment plan for emitting light from the light sources 22 with the controller 100 to eliminate the determined pathogen based on the determined contamination level. The treatment plan may include a determination of at least one wavelength in the range of 185 nm and 405 nm at which to emit the light, at least one intensity at which to emit the light, and at least one duration of time at which to emit the light from the plurality of light sources in accordance with the treatment plan. For example, the system may include an algorithm which determines an amount of energy (e.g., expressed as μWcm²) and associated time required to kill a predetermined virus in the space based on the pathogen table 111. The method may include factoring in a size of the space which may either be detected by the space size sensor 51, or may be manually entered into the controller 100 by the operator as part of the algorithm for determining energy required for the specific space. A safety factor, such as 2, may be introduced into the calculation to ensure that a sufficient amount of energy is employed to eliminate the pathogen. The method further includes 508 emitting light in the space from the at least one of the plurality of light sources 22 in accordance with the treatment plan.

During treatment under the treatment plan, the method may pull data from the various sensors, and modify the treatment plan to improve an efficiency of killing the pathogen and/or provide improved safety. For example, as illustrated in FIG. 5, the method may further include 600 measuring a radiometric energy coming from the at least one light source 22 with a radiometric sensor 47, and 602 modifying the treatment plan based on the measured radiometric energy. For example, the radiometric sensor 47 may determine that light is being emitted at a first radiometric energy from one of the light sources 22, but the controller 100 may determine that the pathogen may be eliminated more quickly if light is emitted at a second radiometric energy, and thus the controller 100 may change a wavelength of light being emitted from the light source 22.

As illustrated in FIG. 6, the method may further include 606 measuring an environmental factor, such as a humidity or temperature in the space with an environment sensor 40, and 608 adjusting the treatment plan based on the measured environment factor. For example, the controller 100 may measure a high temperature in the space, and determine that as a result of the temperature, the pathogen may be killed faster if light is emitted at a different intensity.

As illustrated in FIG. 7, the method may further include 608 detecting the presence of an occupant within the space with an occupant sensor, such as a motion sensor 36, and 610 deactivating at least one of the light sources 22 in response to the detection of the occupant. Alternatively, the system may be configured to 612 change a wavelength (or other parameter such as intensity) of light emitted from the at least one light source 22 as a result of the detection of the occupant. For example, the system may be configured to change the wavelength of light being emitted from 253.7 nm (approx. 254 nm) to 222 nm in order to provide a more safe wavelength of light being emitted during the presence of the occupant. The system may further be configured to 614 revert back to the original wavelength after the occupant is no longer detected by the sensor. According to an aspect, only light sources 22 that are capable of emitting light at the occupant are changed in this manner, while other light sources 22 that are not capable of emitting light at the occupant remain activated/emit light at their original wavelength.

As another example, an approx. 254 nm light may be emitted in a space that is free from occupants, and the treatment plan may require a specific duration of time to emit light at approx. 254 nm in order to reach a specific energy level in order to satisfactorily disinfect the room for a predetermined pathogen. At a point at which 80% of the calculated dose is emitted, a motion sensor 36 may detect the presence of an occupant in the space. Accordingly, the controller 100 may employ the algorithm and instruct one or more of the light sources 22 to emit light at a lower, safer wavelength, such as 222 nm. As part of this process, the controller 100 is configured to calculate a new time duration and/or intensity to emit light in order to reach the target at the new wave length via the algorithm. It should be appreciated that under this scenario, although a longer duration of time may be required than while the light sources 22 operated at the first frequency, the system is able to adapt to still reach the target treatment. Because the algorithm is configured to continuously adapt to changing conditions, the system is able to always calculate how to reach the target in the quickest possible manner.

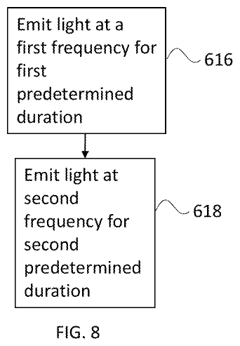

The established treatment plan in accordance with the method may be adapted to a specific schedule of occupiers of the space to be treated. For example, as illustrated in FIG. 8, the treatment plan may include 616 emitting light from at least one of the light sources 22 at a first wavelength that is safe for occupiers to be exposed to for predetermined period of time while the occupiers are present, and then 618 switching to a different wavelength that is less safe for occupiers to be exposed to for a predetermined period of time at which it is known in advance that occupiers will be out of the space. As part of this operation, the method may include activating one or more electronic door locks 37 to prevent occupiers from entering while light is being emitted at unsafe parameters at the second wavelength. The method may further include deactivating the one or more door locks 37 when the light source 22 is deactivated or switched to a safer wavelength.

It should be appreciated that the treatment plan may be modified any numbers of times, and may change any number of factors based on any number of readings. For example, the treatment plan may change a wavelength based on both a detection of an occupant and a detection of particulate during the same treatment cycle.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

Many modifications and variations of the present invention are possible in light of the above teachings and may be practiced otherwise than as specifically described. In addition, the reference numerals are merely for convenience and are not to be read in any way as limiting. Note that not all of the activities described above in the general description or the examples are required, that a portion of a specific activity may not be required, and that one or more further activities may be performed in addition to those described. Still further, the orders in which activities are listed are not necessarily the order in which they are performed. The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Furthermore, certain features are, for clarity, described herein in the context of separate embodiments, may also be provided in combination in a single embodiment. Conversely, various features that are, for brevity, described in the context of a single embodiment, may also be provided separately or in any sub combination.

What is claimed:

1. A method for sanitizing a space, comprising:
positioning a plurality of light sources in a space, each of the light sources configured to emit ultraviolet light in a wavelength range between 185 nm and 405 nm;
determining at least one pathogen which is desired to be killed in the space;
determining a contamination level of the pathogen in the space with at least one sensor;
determining a treatment plan for emitting light from at least one of the plurality of light sources with a controller to eliminate the at least one determined pathogen based on the determined contamination level, wherein the treatment plan includes a determination of at least one wavelength in the range of 185 nm and 405 nm at which to emit the light, at least one intensity at which to emit the light, and at least one duration of time at which to emit the light from the at least one of the plurality of light sources to kill the pathogen; and
emitting light in the space from the at least one of the plurality of light sources in accordance with the treatment plan.

2. A method as set forth in claim 1, wherein the at least one sensor determines an amount of a particulate associated with the determined pathogen in air in the space.

3. A method as set forth in claim 1, further including measuring radiometric energy coming from the at least one light source with a radiometric sensor and modifying the treatment plan based on the measured radiometric energy.

4. A method as set forth in claim 1, further including measuring an environmental factor including at least one of humidity and temperature in the space, and modifying the treatment plan based on the measured environmental factor.

5. A method as set forth in claim 1 further including detecting the presence of an occupant in the space with an occupant sensor, and deactivating at least one of the light sources or changing a wavelength of at least one of the light sources from a first wavelength to a second wavelength in response to the detected presence of the occupant in the space.

6. A method as set forth in claim 5, further including re-activating the at least one of the light sources or reverting back to the first wavelength in response to a detection that the occupant is no longer present in the space with the sensor.

7. A method as set forth in claim 5 wherein at least one of the plurality of light sources remains on in a region of the space that is remote from the detected occupant.

8. A method as set forth in claim 1 wherein determining the treatment plan includes establishing a first predetermined duration of time at which to emit the light at a first wavelength, and a second duration of time at which to emit the light at a second wavelength.

9. A method as set forth in claim 1, further including activating at least one electronic door lock in response to the light being emitted from the at least one light source at a predetermined wavelength to prevent occupants from entering the room.

10. A method as set forth in claim 9, further including deactivating the electronic door lock in response to the light source no longer being admitted at the predetermined wavelength.

11. A method as set forth in claim 1, wherein the at least one sensor includes a plurality of sensors, wherein the plurality of light sources are positioned on a plurality of light assemblies, wherein each of the light assemblies includes at least one of the sensors positioned thereon, and wherein the plurality light assemblies are electrically connected with one another for sharing sensor readings among the light assemblies.

12. A method for sanitizing a space, comprising:
positioning at least one light source in a space, the at least one light source configured to emit ultraviolet light in a wavelength range between 185 nm and 405 nm;
emitting light in the space from the at least one light source at a first wavelength in the range between 185 nm and 405 nm;
detecting a condition in the space with a sensor;
emitting light in the space from the at least one of the plurality of lights sources at a second wavelength that is different than the first wavelength in the range between 185 nm and 405 nm in response to the detection of the condition in the space with the sensor.

13. A method as set forth in claim 12 wherein the at least one sensor is an occupant sensor configured to detect the presence of an occupant in the space, wherein the condition detected is the presence of an occupant in the space with the occupant sensor.

14. A method as set forth in claim 13 wherein the at least one light sources includes a plurality of light sources, and wherein at least one of the plurality of light sources remains at the first wavelength while the wavelength of the other of the light sources is changed in response to the detection of the occupant.

15. A method as set forth in claim 12 wherein the second wavelength is 222 nm.

16. A method as set forth in claim 15 wherein the first wavelength is approximately 254 nm.

17. A method as set forth in claim 12, wherein the at least one sensor determines an amount of a particulate in air in the space.

18. A method as set forth in claim 12, wherein the at least one sensor is a radiometric sensor configured to measure a radiometric energy.

19. A method as set forth in claim 13 further including positioning at least one electronic door lock in the room, and activating the electronic door lock in response to the detection of an occupant with the sensor.

20. A method as set forth in claim 19 further including deactivating the electronic door lock in response to the occupant no longer being detected by the sensor.
